(19) United States
(12) Patent Application Publication
(10) Pub. No.: US 2025/0041556 A1
(43) Pub. Date: Feb. 6, 2025

(54) PERSONAL SPACE POD APPARATUS

(71) Applicant: Fernando Zarate, Chicago, IL (US)

(72) Inventor: Fernando Zarate, Chicago, IL (US)

(21) Appl. No.: 18/229,342

(22) Filed: Aug. 2, 2023

(51) Int. Cl. A61M 21/00; A47C 21/00; A47C 21/04; A47C 27/00

(52) U.S. Cl. CPC A61M 21/0094; A47C 21/003; A47C 21/042; A47C 27/002

(57) ABSTRACT

A personal space pod apparatus for providing a secluded rest area which can be incorporated into public spaces includes a main frame having a bounded interior area, a doorway that permits access to the interior area, and an electronic user interface that communicates electronically with a user's electronic devices. The electronic user interface can be programmed to provide information about the availability of the personal space pod and the amount of time which the user is using the personal space pod. The personal space pod may include a television and sound system, an array of lighting settings, and climate control, all of which may have settings the user can choose based on their personal preferences. The pods may be incorporated into public areas such as airports, hospitals, universities, or offices.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to capsule sleeping pods and more particularly pertains to a new capsule sleeping pod for providing a secluded rest area which can be incorporated into public spaces.

### (2) Description of Related Art Including Information Disclosed Under 37 CFR 1.97 and 1.98

The prior art relates to capsule sleeping pods. The prior art discloses capsule pod sleeping chambers with various amenities and environmental controls. For example, some prior art sleeping pods include short-term, modular resting facilities which may be erected in existing transportation terminal structures such as airports, bus terminals, and train stations. Other prior art examples disclose capsule sleeping chambers with televisions, air filters, and lighting devices. However, the prior art lacks a disclosure for a sleeping pod having the capability of connecting with a user's personal electronic devices for short-term reservations or for providing the user with setting controls over various amenities of the sleeping pod.

### BRIEF SUMMARY OF THE INVENTION

An embodiment of the disclosure meets the needs presented above by generally comprising a personal space pod with a main frame having a bounded interior area, a doorway that permits access to the interior area, and an electronic user interface that communicates electronically with a user's electronic devices. The electronic user interface can be programmed to provide information about the availability of the personal space pod and the amount of time which the user is using the personal space pod. The personal space pod may include a television and sound system, an array of lighting settings, and climate control, all of which may have settings the user can choose based on their personal preferences. The pods may be incorporated into public areas such as airports, hospitals, universities, or offices.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a personal space pod apparatus according to an embodiment of the disclosure.

FIG. 2 is a front view of an embodiment of the disclosure.

FIG. 3 is a top view of an embodiment of the disclosure.

FIG. 4 is an in-use view of an embodiment of the disclosure.

FIG. 5 is a block diagram view of an embodiment of the disclosure.

### DETAILED DESCRIPTION OF THE INVENTION

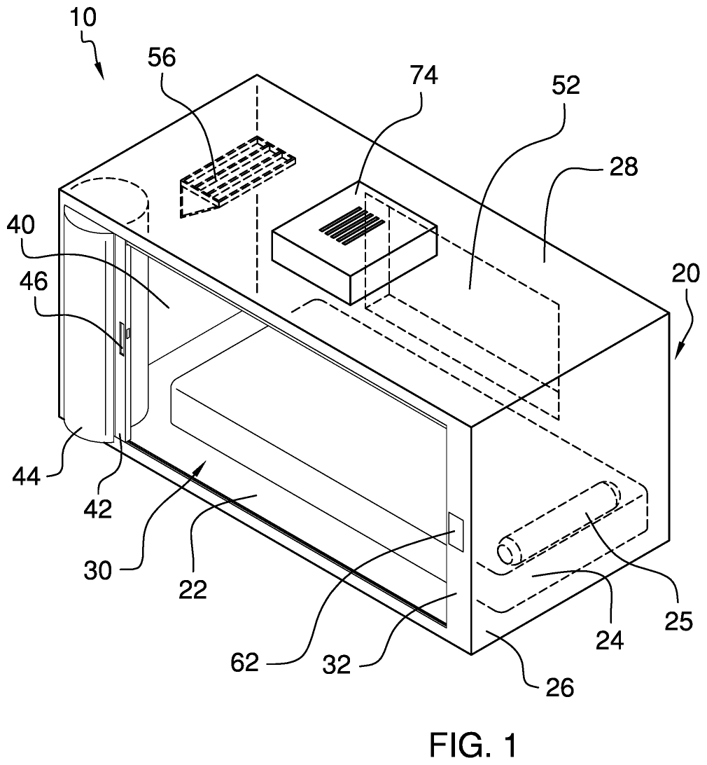

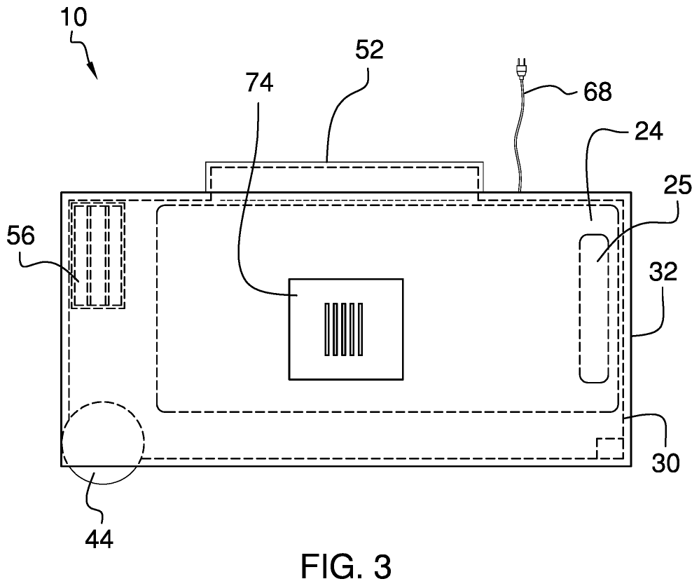

With reference now to the drawings, and in particular to FIGS. 1 through 5 thereof, a new capsule sleeping pod embodying the principles and concepts of an embodiment of the disclosure and generally designated by the reference numeral 10 will be described.

As best illustrated in FIGS. 1 through 5, the personal space pod apparatus 10 generally comprises a main frame 20 having a bounded interior area 30, a doorway 40 configured to permit access to the interior area 30, and an electronic user interface 60 configured to communicate with an electronic device 90 of a user.

The main frame 20 includes a base 22 for supporting a mattress 24. A peripheral wall 26 extends upwardly from the base 22. A ceiling panel 28 is opposite the base 22 from the peripheral wall 26. The main frame 20 has an interior area 30 that is bound by the base 22, the peripheral wall 26, and the ceiling panel 28.

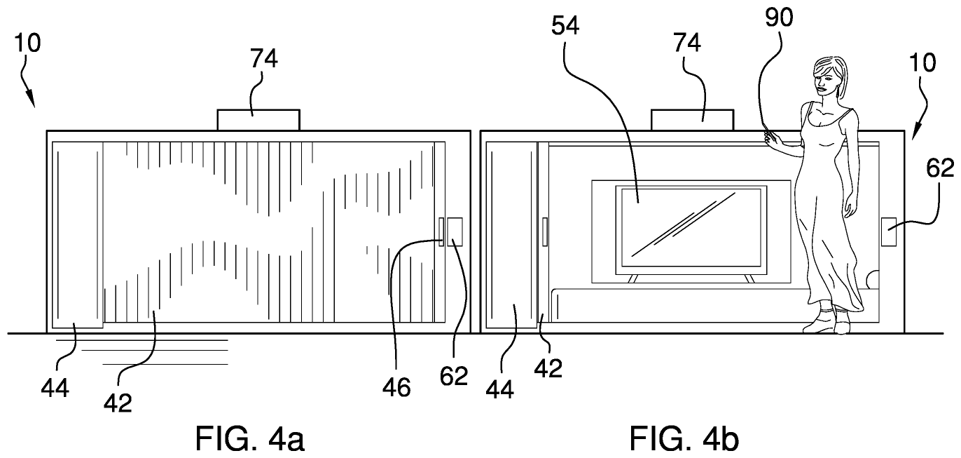

A doorway 40 is positioned in the peripheral wall 26. The doorway 40 is configured to permit access to the interior area 30. In some embodiments, the doorway 40 includes a roller door 42 configured to extend and retract across the doorway 40. As shown in FIG. 4a, the roller door 42 is configured to extend to a closed position thereby preventing access to the interior area 30. As shown in FIG. 4b, the roller door 42 is also configured to retract to an open position thereby permitting access to the interior area 30. The roller door may further include a lock (not pictured) accessible from the interior area 30. With the lock, a user can close and lock the roller door 42 from within the interior area 30 to ensure security and privacy.

The doorway 40 may further include a roller door storage area 44 that is enclosed within the peripheral wall 26 proximate the doorway 40. The roller door storage area 44 is generally configured to enfold the roller door 42 when the roller door is in the closed position. In some embodiments, the roller door 42 includes a handle 46 accessible from the interior area 30 and from the exterior of the main frame 20. In such embodiments, the handle 46 may be accessible while the roller door 42 is in the closed position, as depicted in FIG. 1. In other embodiments, the roller door 42 may be configured to extend and retract from the roller door storage area 44 using an electronically or hydraulically powered motor (not shown) or any other reasonable means. Some embodiments may include multiple means for transitioning the roller door 42 between the open and closed positions.

A plurality of comfort modifications may be housed within the interior area 30. For example, a cutout 52 may be formed along a portion of the peripheral wall 26. In some embodiments, the cutout 52 is sized such that the cutout 52 is configured for supporting a television 54 within the interior area 30. A plurality of charging ports 58 may also be positioned in the peripheral wall such that the charging ports 58 are exposed within the cutout 52.

The mattress 24 may be placed upon the base 22. In some embodiments, the mattress 24 is encased in a plastic coating. A pillow 25 may be provided, for example located on said mattress 24. The pillow 25 may also be encased in a plastic coating. In some embodiments, the base 22, and accordingly the main frame 20, may be sized such that the base 22 is configured to support a twin-sized mattress 24. In other embodiments, the base 22, and accordingly the main frame 20, may be sized such that the base 22 is configured to support other sizes of mattresses 24. With these varying size options available, the personal space pod apparatus 10 may be configured for use by a single user, or for shared use by multiple users. The varying size options may also provide additional choices for a user who is most comfortable resting on a twin-sized mattress, or on a larger mattress.

In some embodiments, the base 22 has an elevated area (not shown) configured to support said mattress 24. The elevated area is raised vertically from said base 22 and the mattress 24 is positionable on said elevated area. The elevated area may provide increased comfort for the user, for example by providing better temperature control for the mattress 24 because the mattress 24 is further elevated from the ground. The elevated base may also provide improved support for the mattress 24, increasing the comfort of the user. The elevated area may also increase the cleanliness of the interior area 30, for example because the user is not walking on the elevated area and tracking dirt or grime from their shoes onto the elevated area where the mattress 24 is positioned.

Some embodiments may also include a shelving unit 56 attached to said peripheral wall 26. The shelving unit 56 is configured to support a personal belonging (not shown) of said user. For example, the shelving unit 56 may include a platform for the user to rest their suitcase or bag. The shelving unit 56 could also include a hanger rod or hook from which the user can hang their personal belonging. The shelving unit 56 is accessible from within the interior area 30 so that the personal belonging is secured while the user rests or relaxes within the personal space pod apparatus 10.

An electronic user interface 60 is mounted within an exterior face 32 of said peripheral wall 26. The electronic user interface 60 is configured to electronically communicate with an electronic or mobile device 90 of a user. The electronic user interface 60 may be configured to electronically communicate with any personal electronic or mobile device 90, such as a mobile phone (as shown in FIG. 4b) a tablet, a laptop, or any other personal computing device.

Embodiments of the electronic user interface 60 include a touch screen panel 62 that is positioned on said exterior face 32, for example adjacent to said doorway 40. The touch screen panel 62 may alternatively be positioned on the exterior face 32 in any location which the user may conveniently access from outside the main frame 20. The touch screen panel 62 is configured to display a user interface display (not shown) of said electronic user interface 60. For example, the touch screen panel 62 may display a user interface display that mirrors or replicates a display screen on the mobile device 90 once the mobile device 90 connects with the electronic user interface 60. A user may also interact directly with the touch screen panel 62 via the user interface display if the user does not have a mobile device 90 that is compatible with the electronic user interface 60 or that is otherwise unable to connect with the electronic user interface 60.

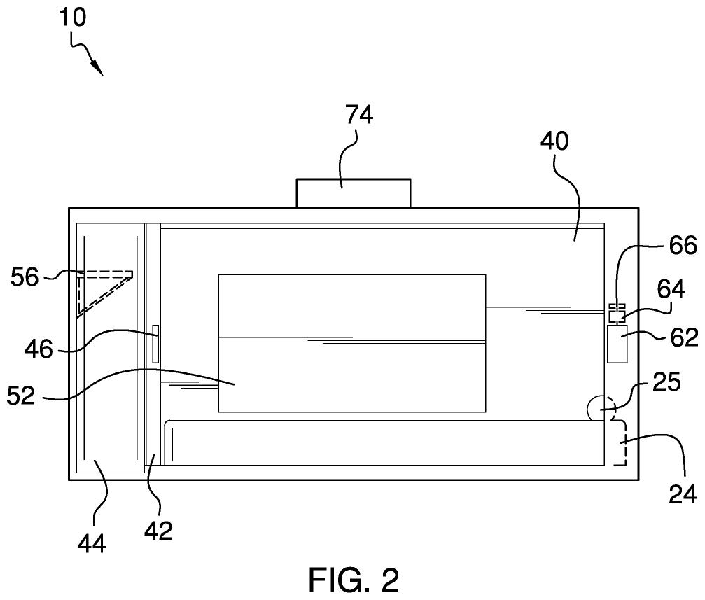

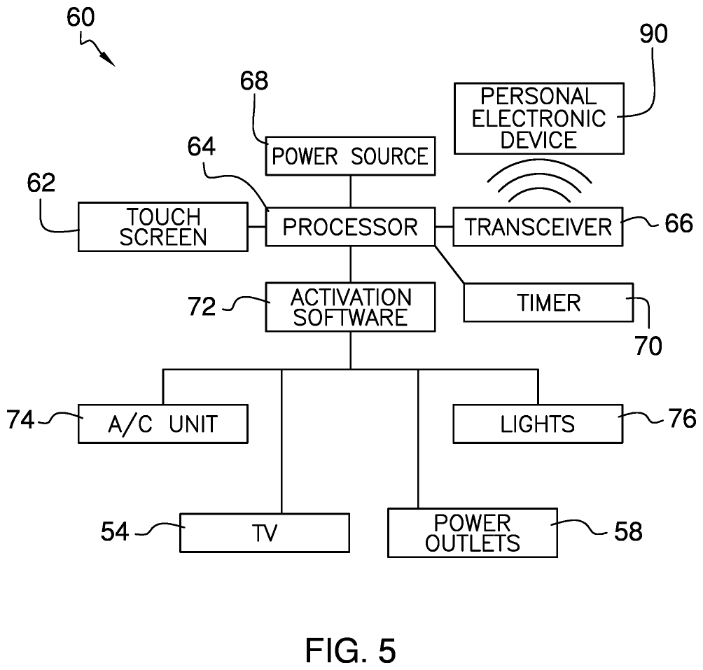

As shown in FIGS. 2 and 5, a processer 64 is electronically coupled with said touch screen panel 62. A transceiver 66 is electronically coupled with said processer 64. The transceiver 66 is generally configured to communicably couple said touch screen panel 62 with said mobile device 90. A power source 68 is electronically coupled with said processor 64.

Embodiments may also include a timer 70 that is electronically coupled with said processor 64. The timer 70 is configured to measure a usage time of said user. The usage time may be measured from a check-in time, for example when said mobile device 90 electronically couples with said transceiver 66, to a check-out time, for example when said mobile device 90 electronically decouples from said transceiver 66. In some embodiments, the transceiver 66 is also configured to communicably couple with an internet or local area network. For example, if the personal space pod is installed in an airport, the transceiver 66 may communicate with that airport's internet. In such embodiments, the user may be able to reserve a personal space pod apparatus 10 remotely, and the usage time may be measured for the period of time which the user has reserved the personal space pod apparatus. This is only one example and should not be construed to limit the disclosure to embodiments within airports or connected to local networks via the internet or wi-fi. Any appropriate network connection may be used.

An activation software 72 is downloaded on said processor 64. The activation software 72 is configured to activate and operate a plurality of functional elements. For example, the activation software 72 may activate and operate the lock (not shown) on the doorway 40. In embodiments where the roller door 42 is coupled to a motor (not shown) that electronically or hydraulically powers the roller door 42, the activation software 72 may activate and operate that motor (not shown), or the roller door 42 itself. The activation software 72 may also activate and operate the plurality of charging ports 58 or the television 54.

The personal space pod apparatus 10 may also include a climate control unit 74 such as an air conditioning filtration unit. For example, the climate control unit 74 may be physically attached to the ceiling panel 28 and electronically coupled with the activation software 72. The climate control unit 74 is generally configured to regulate an ambient temperature and an air flow level within the interior area 30. Using the user interface display (not shown), the user may adjust settings for the climate control unit 74, for example to raise or lower the ambient temperature or air flow level.

The personal space pod apparatus 10 may also include a lighting system 76 having a plurality of light settings. For example, the lighting system 76 may emit a plurality of brightness levels and a plurality of lighting colors, based on settings chosen by the user via the electronic user interface 60 or the user interface display (not shown).

The personal space pod apparatus 10 may also include a sound system (not shown) having a plurality of volume levels. The sound system (not shown) may be in electronic communication with said television 54 and with said mobile device 90. For example, the sound system (not shown) may emit sound from the television 54 or from an audio file of the mobile device 90. Using the sound system (not shown), the user may enjoy a program on the television 54 or music, podcasts, or other audio files on their personal electronic device 90.

In use, the user may use the touch screen panel 62 or the electronic user interface 60 to check if the personal space pod apparatus 10 is available. If it is, the user may connect their mobile device 90 to immediately begin use, or they may reserve a time for future use. The user need only keep their electronic device 90 connected with the electronic user interface 60 for the time the user wants to utilize the personal space pod apparatus 10. With the personal space pod apparatus 10, the user can rest and relax in a private area within public spaces such as airports, universities, hospitals, or offices.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A personal space pod apparatus, comprising:
a main frame including a base for supporting a mattress, a peripheral wall extending upwardly from said base, and a ceiling panel opposite said base from said peripheral wall, said main frame having an interior area being bound by the base, the peripheral wall, and the ceiling panel;
a doorway being positioned in the peripheral wall, wherein the doorway is configured to permit access to the interior area;
an electronic user interface being positioned on an exterior face of the peripheral wall, the electronic user interface being configured to electronically communicate with an electronic device of a user, the electronic user interface including:
a touch screen panel being positioned on the exterior face, the touch screen panel being configured to display a user interface display of the electronic user interface;
a processer electronically coupled with the touch screen panel;
a transceiver electronically coupled with the processer, the transceiver being configured to operationally connect the touch screen panel with the electronic device; and
a power source being electronically coupled with the processor.

2. The personal space pod apparatus of claim 1, the doorway further comprising:
a roller door configured to extend and retract across the doorway, the roller door being configured to extend to a closed position thereby preventing access to the interior area and being further configured to retract to an open position thereby permitting access to the interior area; and
a roller door storage area being enclosed within the peripheral wall proximate the doorway, the roller door storage area being configured to enfold the roller door when the roller door is in the closed position.

3. The personal space pod apparatus of claim 2, the roller door further comprising a lock accessible from the interior area.

4. The personal space pod apparatus of claim 1, further comprising a cutout being sized such that the cutout is configured for supporting a television within the interior area.

5. The personal space pod apparatus of claim 1, further comprising a plurality of charging ports being positioned in the peripheral wall and being accessible from the interior area.

6. The personal space pod apparatus of claim 1, further comprising a shelving unit attached to the peripheral wall, the shelving unit being configured to support a personal belonging of the user.

7. The personal space pod apparatus of claim 1, the electronic user interface further comprising an activation software being downloaded on the processor, the activation software being configured to activate and operate a plurality of functional elements, the plurality of functional elements including the user interface display.

8. The personal space pod apparatus of claim 7, the plurality of functional elements further comprising a climate control unit being attached to the ceiling panel, the climate control unit being configured to regulate an ambient temperature and an air flow level within the interior area.

9. The personal space pod apparatus of claim 7, the plurality of functional elements further comprising a lighting system having a plurality of light settings, wherein the lighting system emits a plurality of brightness levels and a plurality of lighting colors.

10. The personal space pod apparatus of claim 1, the electronic user interface further comprising a timer electronically coupled with the processor, the timer being configured to measure a usage time of the user, the usage time being measured from a check-in time when the electronic device operationally connects with the transceiver to a check-out time when the electronic device operationally disconnects from the transceiver.

11. The personal space pod apparatus of claim 1, the base having an elevated area configured to support the mattress, the elevated area being raised vertically from the base.

12. The personal space pod apparatus of claim 11, wherein the mattress is positionable on the elevated area.

13. The personal space pod apparatus of claim 1, the mattress further comprising a plastic coating encasing the mattress.

14. The personal space pod apparatus of claim 1, wherein the electronic device comprises a mobile phone, a tablet, a laptop, or another personal computing device.

15. A personal space pod apparatus, comprising:
a main frame including a base for supporting a mattress, a peripheral wall extending upwardly from the base, and a ceiling panel being opposite the base from the peripheral wall, the main frame having an interior area being bound by the base, the peripheral wall, and the ceiling panel;
a doorway being positioned in the peripheral wall, wherein the doorway is configured to permit access to the interior area, the doorway including:
a roller door configured to extend and retract across the doorway, the roller door being configured to extend to a closed position thereby preventing access to the interior area and being further configured to retract to an open position thereby permitting access to the interior area, the roller door including a lock accessible from the interior area;
a roller door storage area being enclosed within the peripheral wall proximate the doorway, the roller door storage area being configured to enfold the roller door when the roller door is in the closed position;
a plurality of comfort modifications, including:
a cutout being sized such that the cutout is configured for supporting a television within the interior area;
a plurality of charging ports being positioned in the peripheral wall and being exposed within the cutout;
the mattress being located on the base, the mattress being encased in a plastic coating;
a shelving unit attached to the peripheral wall, the shelving unit being configured to support a personal belonging of the user;
an electronic user interface being mounted within an exterior face of the peripheral wall, the electronic user interface being configured to electronically communicate with a mobile device of a user, the electronic user interface including:
a touch screen panel being positioned on the exterior face adjacent to the doorway, the touch screen panel being configured to display a user interface display of the electronic user interface;
a processer electronically coupled with the touch screen panel;
a transceiver electronically coupled with the processer, the transceiver being configured to communicably couple the touch screen panel with the mobile device;
a power source being electronically coupled with the processor;
a timer electronically coupled with the processor, the timer being configured to measure a usage time of the user, the usage time being measured from a check-in time when the mobile device electronically couples with the transceiver to a check-out time when the mobile device electronically decouples from the transceiver;
an activation software being downloaded on the processor, the activation software being configured to activate and operate a plurality of functional elements, the plurality of functional elements including:
an air conditioning filtration unit being attached to the ceiling panel, the air conditioning filtration unit being configured to regulate an ambient temperature and an air flow level within the interior area;
a lighting system having a plurality of light settings, wherein the lighting system emits a plurality of brightness levels and a plurality of lighting colors;
the lock;
the television;
a sound system having a plurality of volume levels, the sound system being in electronic communication with the television and with the mobile device; and
the plurality of charging ports.
